(19) United States
(12) Patent Application Publication
(10) Pub. No.: US 2025/0031679 A1
(43) Pub. Date: Jan. 30, 2025

(54) SCREENING METHOD OF PROBIOTIC AND ACTIVE SUBSTANCE FOR PROMOTING REPRODUCTIVE HEALTH AND USE

(71) Applicants: Xuzhou Medical University, Xuzhou (CN); XUZHOU CENTRAL HOSPITAL, Xuzhou (CN)

(72) Inventors: Zuobin ZHU, Xuzhou (CN); Conghui HAN, Xuzhou (CN); Yalun WU, Xuzhou (CN); Ying LI, Xuzhou (CN)

(73) Assignees: Xuzhou Medical University, Xuzhou (CN); XUZHOU CENTRAL HOSPITAL, Xuzhou (CN)

(21) Appl. No.: 18/367,354

(22) Filed: Sep. 12, 2023

(30) Foreign Application Priority Data
Jul. 24, 2023 (CN) 2023109059156

(51) Int. Cl. A01K 67/033; A61K 49/00

(52) U.S. Cl. CPC A01K 67/033; A61K 49/0008; A01K 2207/20; A01K 2227/703; A01K 2267/03

(57) ABSTRACT

The present disclosure provides a method for screening a probiotic strain in promoting reproductive health by using a reproductive disorder model of Caenorhabditis elegans. Cyclophosphamide (CTX) drug intervention is applied to the C. elegans to destroy a normal reproductive system of the C. elegans, thereby obtaining the reproductive disorder model of the C. elegans. Strains capable of significantly improving a reproductive capacity of individuals with reproductive disorders are selected from 3,985 kinds of gene-knockout strains in a Keio gene-knockout library of Escherichia coli; and homologous gene knockout is conducted on a probiotic strain E. coli Nissle 1917 by gene editing, and a probiotic strain with a potential to promote the reproductive health is selected.

### CROSS REFERENCE TO RELATED APPLICATION

This patent application claims the benefit and priority of Chinese Patent Application No. 2023109059156 filed with the China National Intellectual Property Administration on Jul. 24, 2023, the disclosure of which is incorporated by reference herein in its entirety as part of the present application.

### TECHNICAL FIELD

The present disclosure relates to the field of biotechnology, in particular to a method for constructing a reproductive disorder model of Caenorhabditis elegans, and use of the reproductive disorder model in screening a probiotic for promoting reproductive health.

### BACKGROUND

Caenorhabditis elegans is a small nematode living in soil as well as a typical model organism that feeds on bacteria. C. elegans has clear genetic background, transparent body, simple structure, short life cycle, and complete genome sequencing. Therefore. C. elegans is widely used in various fields such as toxicology, genetics and developmental biology, ethology and neurobiology, aging and longevity, human genetic diseases, interactions between pathogens and biological organisms, and drug screening in recent years. At present. C. elegans has been widely used in aging research, drug toxicity, drug screening, and construction of various biological models. For example: CN115067283A disclosed a method for constructing an obesity model using C. elegans; CN108670961A disclosed a method for constructing a drunkenness model using C. elegans, and use of the drunkenness model in quickly screening a hangover-relief plant. In the prior art, cyclophosphamide (CTX) is a common anti-tumor drug clinically, and has been clinically used for years. However, the CTX also causes severe damages to the reproductive function while exerting tumor therapeutic effects. Clinically, CTX has been used in recent years so widely that it renders a gradual increase in infertility patients. It is an effective way to improve a clinical application value of the CTX and reduce the complications of clinical reproductive disorder by researching drugs that reduce the reproductive toxicity of CTX Accordingly, it is of practical significance to construct a reproductive disorder model of C. elegans using the CTX.

Genetically engineered probiotic strains are currently a hot field in the clinical application of intestinal microbes. Escherichia coli Nissle 1917, as a well-studied probiotic strain, was isolated by German bacteriologist Alfred Nissle in 1917. Currently, there is a finished drug “Mutaflor” made of E. coli Nissle 1917 as a main component. E. coli Nissle 1917 is a potential therapeutic probiotic strain, and can colonize in human intestinal tract to protect and repair the intestinal mucosa, thereby preventing the intestinal tract from being attacked by pathogenic bacteria. In addition, E. coli Nissle 1917 also participates in immune regulation of the host and targets tumors, making this strain a desirable drug carrier with an excellent research prospect. Meanwhile, the reproductive health of human beings is not optimistic nowadays. Global research data show that human reproductive capacity has a downward trend, where the quality of male semen has decreased significantly, while the rate of female infertility has increased significantly. As a result, the human reproductive health is formidably challenged.

In view of this, it is of great significance for the development of probiotics or drugs that can improve animal reproductive dysfunction in patients by applying the reproductive disorder model of C. elegans to the screening of genetically engineered probiotic strains.

### SUMMARY

An objective of the present disclosure is to provide a method for constructing a reproductive disorder model using Caenorhabditis elegans. The present disclosure further provides use of the reproductive disorder model of the C. elegans in studying reproductive-related mechanisms, screening a probiotic for promoting reproductive health, and developing a related drug.

To achieve the above objective, the present disclosure provides the following technical solutions:

The present disclosure provides a method for constructing a reproductive disorder model, including the following steps:
obtaining synchronized L1-stage Caenorhabditis elegans; and
putting the L1-stage C. elegans in an S broth containing 0.5 mg/mL to 2.0 mg/ml of cyclophosphamide (CTX) to allow culturing for 48 h to 96 h to complete model construction.

In some embodiments, the CTX has a concentration of 1 mg/mL.

In some embodiments, the synchronized C. elegans is obtained by a sodium hypochlorite process.

In some embodiments, the synchronized C. elegans is cultured to hatch eggs to obtain the synchronized L1-stage C. elegans.

In some embodiments, the C. elegans is wild-type N2 C. elegans.

The present disclosure further provides use of the reproductive disorder model in screening a strain capable of promoting reproductive health.

Specifically, the C. elegans model is fed Escherichia coli strains, and E. coli strains capable of improving a reproductive capacity of the C. elegans are selected.

In some embodiments, the E. coli strains include 3,985 kinds of E. coli strains in a Keio gene-knockout library of E. coli.

In some embodiments, the wild-type N2 C. elegans is fed the E. coli strains capable of improving the reproductive capacity of the C. elegans, and an E. coli strain capable of significantly improving the reproductive capacity of the wild-type N2 C. elegans is selected.

In other embodiments, a homologous gene in a probiotic strain Nissle 1917 is knocked out to obtain a probiotic strain capable of promoting the reproductive health, where the homologous gene corresponds to a knockout gene of the strain capable of improving the reproductive capacity of the C. elegans.

The present disclosure further provides use of the constructed reproductive disorder model in verifying single gene deletion of E. coli in a reproductive system.

The embodiments of the present disclosure have the following beneficial effects:

(1) In the present disclosure, C. elegans, a recognized model organism, is readily available, and can effectively reduce the test cost. The C. elegans shows a short growth cycle and can be mass-proliferated in a short period of time to obtain tens of thousands of nematodes for testing, thus ensuring sufficient sample size, reducing test errors, and guaranteeing the accuracy of evaluation results. High-throughput screening tests can be conducted to efficiently obtain test results, with a greatly shortened test cycle.

(2) In the present disclosure, the animal model of C. elegans is stable and reproducible, and can be used as an animal model of reproductive disorder for research on mechanisms related to reproductive capacity and screening of probiotics that are capable of promoting reproductive health. Meanwhile, the model has simplicity, low cost, and short period.

(3) In the present disclosure, the modeling method of the reproductive disorder model of the C. elegans may greatly reduce the reproductive capacity of the C. elegans.

(4) In the construction method of the present disclosure, the modeling starts at an L1 stage of C. elegans, which may better destroy the reproductive system of the C. elegans to effectively simulate a low reproductive state of this nematode. Moreover, after the synchronization of C. elegans is completed by the sodium hypochlorite process, a growth cycle of this nematode is synchronized by incubation in a 20° C. incubator, such that interference such as pollution removal and growth cycle synchronization are realized.

(5) In the present disclosure, after construction of the reproductive disorder model, a strain capable of improving the reproductive capacity of the C. elegans is selected based on a Keio gene-knockout library of E. coli. Furthermore, the probiotic Nissle 1917 is modified for final selection of a probiotic that can promote the reproductive health of nematodes. The Nissle 1917 is a widely used desirable probiotic, endowing this method with highly wide application prospect and value.

(6) In the present disclosure, the reproductive disorder model of C. elegans is established using CTX. From 3,985 mutated genetically engineered strains of E. coli, a genetically mutated strain that can significantly improve the reproductive capacity of nematodes is selected. In addition, related homologous genes in the human probiotic strain Nissle 1917 are transformed using clustered regularly interspaced short palindromic repeats (CRISPR) and CRISPR associated protein 9 (CRISPR/Cas9) technology. This provides candidate probiotic strains that can treat reproductive disorders and protect reproductive functions for infertile patients or for the use of adjuvant clinical anti-tumor drugs.

### BRIEF DESCRIPTION OF THE DRAWINGS

FIG. 1 shows a flowchart of constructing a reproductive disorder model and screening a probiotic for reproductive health;

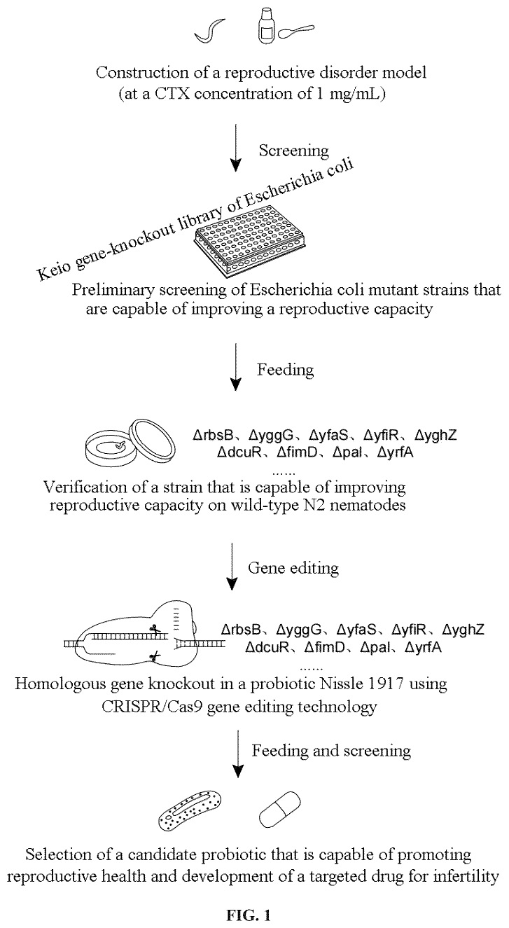

FIG. 2 shows impact of different concentrations of CTX on the number of reproduced C. elegans;

FIG. 3 shows effect of different treatment durations of CTX on the number of reproduced C. elegans. (±SD) (*p<0.05, **p<0.01 vs. blank group);

FIG. 4 shows a peak diagram of effect of an E. coli mutant strain on the reproductive capacity of the reproductive disorder model of C. elegans; and

FIG. 5 shows a histogram illustrating effect of the E. coli mutant strain on the reproductive capacity of the reproductive disorder model of C. elegans.

### DETAILED DESCRIPTION OF THE EMBODIMENTS

The present disclosure provides a method for constructing a reproductive disorder model, including the following steps:
obtaining synchronized L1-stage C. elegans; in some embodiments, after the ion of C. elegans is synchronized by a sodium hypochlorite process, the synchronized C. elegans is cultured at 20° C. to hatch eggs to obtain the synchronized L1-stage C. elegans; and
treating L1-stage C. elegans with CTX at a final concentration of 0.5 mg/mL to 2.0 mg/mL, and conducting culture at 20° C. for 48 h to 96 h to complete model construction; in some embodiments, the L1-stage C. elegans is placed in an S broth containing CTX.

In some embodiments, the model construction is carried out by incubation at 20° C. for 72 h.

In some embodiments, the CTX has a final concentration of 1.0 mg/mL.

In some embodiments, the C. elegans is wild-type N2 C. elegans.

S broth: (1) S basal medium: 5.9 g of NaCl and 50 mL of 1 M potassium phosphate are added with water to 1 L, autoclaved, and added with 1 mL of 5 mg/mL cholesterol after cooling; 10 mL of 1 M potassium citrate, 10 mL of trace metal solution (sterile), 3 mL of 1 M CaCl₂ (sterile), and 3 mL of 1 M MgSO₄ (sterile) are added into 977 mL of the (1).

A formula of the trace metal solution includes: 1.86 g of EDTA-Na₂, 0.69 g FeSO₄·7H₂O, 0.2 g of MnCl₂·4H₂O, 0.29 g of ZnSO₄·7H₂O. and 0.025 g of CuSO₄·5H₂O per 1 L solution.

The present disclosure further provides use of the reproductive disorder model constructed by the construction method in screening a probiotic strain that is capable of promoting reproductive health.

Specifically, a C. elegans model is fed E. coli strains, and E. coli strains capable of improving a reproductive capacity of the C. elegans are selected.

In some embodiments, the E. coli strains include 3,985 kinds of E. coli strains in a Keio gene-knockout library of E. coli.

Specifically, the reproductive disorder model is fed the E. coli in the Keio gene-knockout library. The E. coli is cultured in a shaker at 37° C. for 12 h, centrifuged at 6,000 rpm and inoculated on a Nematode Growth Medium (NGM) plate. Individual C. elegans adults with reproductive disorder are individually picked into an NGM with E. coli mutants. Adults of C. elegans with reproductive disorder are individually transferred to an NGM with a new E. coli mutant daily until the C. elegans ceases to lay eggs. A total number of hatched eggs of each C. elegans is calculated, and a strain that is capable of improving the reproductive capacity of C. elegans is selected.

The NGM is prepared as follows: (1) 2.5 g of peptone, 3 g of NaCl, and 17 g of agar, and a balance of water to 975 mL are autoclaved, and cooled to 55° C. to 60° C.; (2) a phosphate (K₂HPO₄—KH₂PO₄) buffer (pH=6) including 35.6 g of K₂HPO₄, 108.3 g of KH₂PO₄, and a balance of water to 1 L are autoclaved for long-time storage; (3) 1 mol/L MgSO₄ solution is autoclaved for long time storage; (4) 1 mol/L CaCl₂ solution is autoclaved for long time storage; and (5) 5 mg/mL cholesterol-ethanol solution (without autoclaving) is prepared for long time storage. The sterilized (1) is added with 25 mL of the (2), 1 mL of the (3), 1 mL of the (4), and 1 mL of the (5).

In some embodiments, the use further includes: feeding the wild-type N2 C. elegans with the E. coli strains that are capable of improving the reproductive capacity of the C. elegans, and selecting an E. coli strain that is capable of significantly improving the reproductive capacity of the wild-type N2 C. elegans.

Specifically, the wild-type N2 nematode is verified by feeding the E. coli strain capable of improving the reproductive capacity of the nematode. The selected strain with improved reproductive capacity is cultured in a shaker at 37° C. for 12 h, centrifuged at 6,000 rpm and spotted on an NGM plate. Individual wild-type N2 nematode is individually picked into an NGM with E. coli mutants. Adults of N2 nematode is individually transferred to an NGM with a new E. coli mutant that can improve reproductive capacity until the C. elegans ceases to lay eggs. A total number of hatched eggs of each N2 nematode is calculated. A strain that is capable of significantly improving the reproductive capacity of the wild-type N2 nematode is selected.

In other embodiments, a homologous gene in a probiotic strain Nissle 1917 is knocked out to obtain a probiotic strain capable of promoting the reproductive health, where the homologous gene corresponds to a knockout gene of the strain capable of improving the reproductive capacity of the C. elegans.

Specifically, a homologous gene that is capable of improving the reproductive capacity of C. elegans is knocked out on the probiotic Nissle 1917 by CRISPR/Cas9 technology, and a probiotic capable of promoting reproductive health is selected. The gene-edited probiotic is cultured in a shaker at 37° C. for 12 h, centrifuged at 6,000 rpm and inoculated to an NGM plate. Individual N2 adult with reproductive disorder is individually picked into an NGM with probiotic. Adults of N2 nematode is individually transferred to an NGM with a new probiotic until the C. elegans ceases to lay eggs. A total number of hatched eggs of each N2 nematode is calculated, and a probiotic capable of promoting reproductive health is selected.

The present disclosure further provides use of the reproductive disorder model constructed by the construction method in verifying single gene deletion of E. coli in a reproductive system.

In the present disclosure, unless otherwise specified, all raw materials are commercially available products well known to those skilled in the art.

The technical solutions of the present disclosure will be clearly and completely described below with reference to the examples of the present disclosure. Apparently, the described examples are merely a part rather than all of the embodiments of the present disclosure. All other embodiments obtained by those skilled in the art based on the embodiments of the present disclosure without creative efforts shall fall within the protection scope of the present disclosure.

### Example 1

Establishment of a Reproductive Disorder Model of C. elegans

### 1. Material

Strain: E. coli OP50 was purchased from Caenorhabditis Genetics Center (CGC) in the United States.

C. elegans: wild-type N2 was purchased from CGC, USA.

LB solid medium: 10 g of tryptone, 5 g of yeast extract, 10 g of NaCl, and 17 g of agar, diluted to 1 L. and autoclaved.

LB broth: 10 g of tryptone, 5 g of yeast extract, and 10 g of NaCl, diluted to 1 L, and autoclaved.

A preparation method of the Nematode Growth Medium (NGM) included: (1) 2.5 g of peptone, 3 g of NaCl, and 17 g of agar were mixed, added with water to 975 mL, autoclaved, and cooled to 55° C. to 60° C.; (2) a phosphate (K₂HPO₄—KH₂PO₄) buffer (pH=6), 35.6 g of K₂HPO₄, and 108.3 g of KH₂PO₄ were mixed, added with water to 1 L, autoclaved for long time storage; (3) a 1 mol/L MgSO₄ solution, was autoclaved for long time storage; (4) 1 mol/L CaCl₂ solution was autoclaved for long time storage; and (5) 5 mg/mL cholesterol-ethanol solution (without autoclaving), was prepared for long time storage. The sterilized (1) was added with 25 mL of the (2), 1 mL of the (3), 1 mL of the (4), and 1 mL of the (5).

S broth: (1) S basal medium: 5.9 g of NaCl and 50 mL of 1 M potassium phosphate were added with water to 1 L, autoclaved, and added with 1 mL of 5 mg/mL cholesterol after cooling; 10 mL of 1 M potassium citrate. 10 mL of trace metal solution (sterile), 3 mL of 1 M CaCl₂ (sterile), and 3 mL of 1 M MgSO₄ (sterile) were added into 977 mL of the (1).

M9 buffer: 3 g of KH₂PO₄, 6 g of NaHPO₄, 5 g of NaCl, 1 mL of 1M MgSO₄ were mixed with water to make up to 1 L. and autoclaved.

CTX mother solution: 20 mg of CTX was dissolved in 1 mL of sterilized pure water to obtain a 20 mg/mL mother solution, which was prepared for use immediately.

Other materials: sodium hydroxide; 4% sodium hypochlorite solution; kanamycin sulfate; worm picker for nematodes.

2. Culture of E. coli OP50

OP50 monoclonal colonies were cultured by the streaking method on the LB solid medium to obtain strains and stored in refrigerator at 4° C. When being used, the monoclonal strains were picked and cultured in LB broth for 12 h at 37° C. and 130 rpm in a constant-temperature shaker. Alternatively, a cryopreserved OP50 strain was revived and amplified, activated in an LB medium at 37° C., and monoclonal colonies or bacterial solutions were picked and transferred to a new LB medium for amplification. An OD value of the amplified OP50 bacterial solution was adjusted, and the bacterial solution was stored at 4° C. for later use. The OP50 was tested for purity before use, and if there was contamination, the sample was purified and re-cultured.

3. Recovery of C. elegans

The prepared NGM solution was poured into a plate before solidification, and the newly prepared NGM plate was placed on an operating table to air dry and sterilized by ultraviolet light.

The cryopreservation tube containing C. elegans was taken out from the −80° C. refrigerator and placed in a prepared 37° C. water bath to rapidly melt the nematode liquid. The nematode liquid was centrifuged at 3,000 rpm, a supernatant was discarded, and then remaining substances were washed repeatedly with M9 buffer 3 times to remove the cryopreservation solution in the tube. The nematodes were drawn into NGM with a pipette, thus completing the recovery of C. elegans.

4. Subculture of C. elegans

The recovered C. elegans was subcultured using NGM. An old NGM plate that was free from bacterial contamination and contained a large number of nematodes and eggs was selected; a clean blade was sterilized with an alcohol lamp, and a piece of old medium was cut off by the blade and then placed in a new NGM; the nematodes crawled from the old plate to the new plate, and thus the subculture of the nematodes is accomplished.

The C. elegans was mainly cultivated at 20° C. and the incubator was a standard biochemical incubator special for the cultivation of C. elegans.

5. Synchronization in C. elegans

The first stage of synchronization and contamination cleanup was conducted using a sodium hypochlorite synchronization process. A sufficient amount of M9 buffer, 10 mol/L NaOH solution, and 13% NaClO solution were prepared, and then a worm-containing plate was washed with M9 buffer. A worm-containing washing liquid was collected in a sterilized 15 ml centrifuge tube, resuspended with the M9 buffer, and the above operations were repeated until the liquid was clear, and a supernatant was discarded. 0.5 mL of NaOH solution was mixed with 0.5 mL of NaClO solution and 5 mL of sterilized pure water, added to a centrifuge tube containing worms, and the centrifuge tube was shaken gently until most of the worms were broken or dissolved. The centrifuge tube was centrifuged at 3,000 rpm for 1 min, a supernatant was discarded, and remaining substances were washed with M9 buffer. The above operations were repeated about 3 to 4 times until there was no hypochlorous acid smell. On the next day, the eggs hatched to obtain synchronized L1-stage nematodes.

### 6. CTX Treatment

The synchronized L1-stage nematodes were diluted to 20 to 30 nematodes/10 μL. The L1-stage C. elegans was collected in the S broth and cultured in a 96-well plate (365 μL/well). 165 μL of medium, 20 μL of different concentrations of CTX treatment groups, 10 μL of L1-stage nematodes, and 5 μL of OP50 were added in sequence into the 96-well plate, and the nematodes were cultured in a biological incubator at 20° C. for 72 h, where each concentration was set with 10 groups in parallel.

The CTX was set up in three exposure groups from low to high, with concentrations being 500 μg/mL, 1 mg/mL, and 2 mg/mL. At the same time, a blank control was set up.

The nematodes treated with different concentrations of drugs were transferred to NGM, and only one nematode was placed on each medium. The one nematode was picked to a new medium every day with a worm picker. After nematodes produced by this one nematode on the old medium grew to adults, the number of nematodes on the NGM plates with different concentrations of CTX was counted until this one nematode no longer lay eggs. A sum of the number of nematodes exchanged every day was the number of eggs laid and hatched in a whole reproductive cycle of the nematode, and the reproductive capacity of the nematode was determined based on this.

### 7. Data Processing

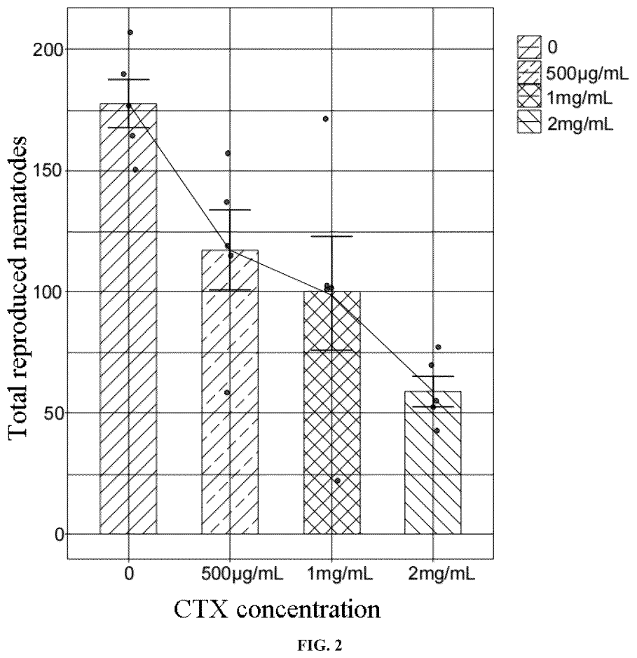

All data were analyzed for significance using Graph Pad 8.0.2 software. Referring to FIG. 2, blank group and optimized modeling group are shown. Compared with the blank group, the optimized modeling group had significantly lower reproductive capacity (P<0.05). Moreover, when the concentration increased from 500 μg/mL to 2 mg/mL, the reproductive capacity of C. elegans decreased with the increase of concentration, showing a concentration-dependent effect. According to statistical analysis. CTX at a concentration of 1 mg/mL could reduce the reproductive capacity of nematodes by 50%, and was therefore used in subsequent construction of the reproductive disorder model of C. elegans.

### Example 2

### CTX Treatment

L1-stage C. elegans was obtained according to the protocol in Example 1, and was treated with the CTX at a concentration of 1 mg/mL according to Example 1. Effect of treatment time on the reproductive capacity of the C. elegans was investigated.

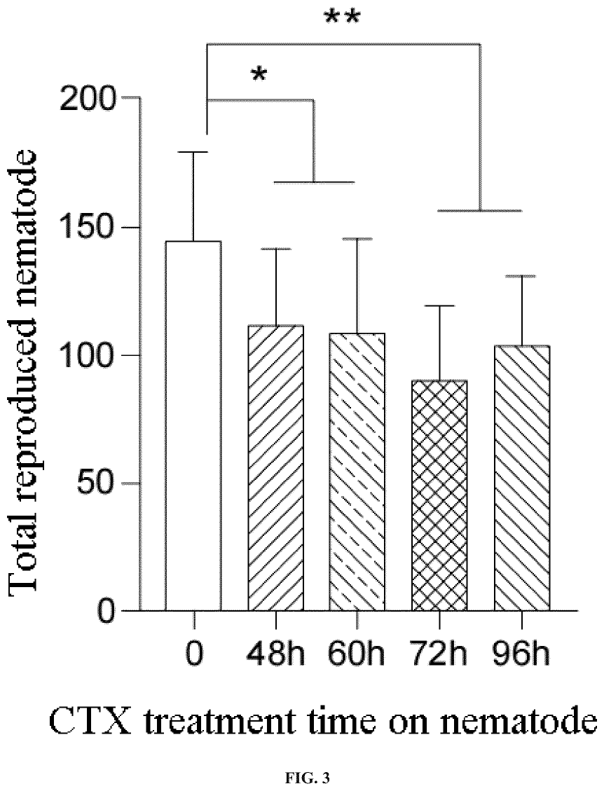

The results are shown in FIG. 3. Compared with the control, it was seen that the total number of reproduced C. elegans could be significantly reduced after 48 h to 96 h treatment. Overall, with an increase of treatment time, the total number of reproduced C. elegans gradually decreased. When treated for 72 h, total reproduced C. elegans was reduced to the minimum; with the prolongation of treatment time, the total number of reproduced C. elegans slightly picked up.

### Example 3

### Experiments on the Differences in Construction of Models for Nematodes at Different Stages of L1 to L4

Nematodes in L1 to L4 stages were used to construct models and the rest of the steps were conducted according to the method in Example 1.

It was found in the experiment that drug treatment at different stages of larvae might significantly reduce the fecundity of nematodes. However, in view of the fact that in the previous experiment, the total number of reproduced C. elegans decreased to the minimum when CTX was applied for 72 h, which was the time from L1-stage nematodes to adults, i.e., the cycle of development and maturation for the nematode germ cells. Moreover, the L1-stage nematodes were relatively easy to accurately identify. The nematodes started to lay eggs after 72 h of treatment, which was also the cycle from germ cell generation to maturity. Therefore, from the perspectives of experimental validity, accuracy, and convenience for large-scale drug screening, the modeling was started at the L1 stage of the C. elegans to simulate a low reproductive state of the C. elegans.

### Example 4

### Experiments on the Differences Between Broth and Solid Medium in Modeling

The model was constructed according to the method in Example 1, and S broth and solid NGM were used separately during CTX treatment.

A method of using the S broth was the same as that in Example 1.

A method of using the solid NGM was to uniformly plate 1 mg/mL of CTX on a surface of the solid NGM.

It tuned out in the experiment that building models with CTX in S broth, in which CTX was easily ingested by C. elegans, makes C. elegans absorb the CTX better. However, when the modeling was conducted on solid NGM, the CTX was slightly soluble in water and could only be dispersed in solid NGM in the form of particles. The C. elegans showed a poor ingestion when cultured on the surface of solid NGM.

### Example 5

The reproductive disorder animal model constructed in Example 1 was taken for selection of strains capable of improving the reproductive capacity of the C. elegans.

1. Determination of Reproductive Capacity of C. elegans

At 20° C. a blank control group and a CTX treatment group were given pure OP50 bacterial solution and 1 mg/mL CTX/OP50 bacterial solution, respectively. Food were supplemented every 24 h, and whether the nematodes survived normally was observed. After treated in S broth for 72 h, the nematodes in the blank group and the CTX treatment group were transferred to NGM with food OP50, and one nematode was placed on each medium. The one nematode was picked to a new medium every day with a worm picker After nematodes produced by this one nematode on the old medium grew to adults, the number of nematodes on NGM plates of the blank group and the CTX treatment group was counted until this one nematode no longer lay eggs. Referring to FIG. 2, in the blank group and the optimized modeling group, the total number of nematodes reproduced in the CTX treatment group at a concentration of 1 mg/mL was reduced by 50% compared with that in the blank control group (P<0.05).

2. Verification of the Keto Gene-Knockout Library of E. coli in Improving the Reproductive Capacity of the Reproductive Disorder Model of C. elegans

The Keio library was purchased from Advanced Biosciences, Keio University (Japan), Nara Institute of Science and Technology (Japan), and Purdue University, which was prepared according to Baba, Tomoya, et al. Construction of Escherichia coli K-12 in-frame, single-gene knockout mutants: the Keio collection. Molecular systems biology, 2006, 2. 1:2006.0008.

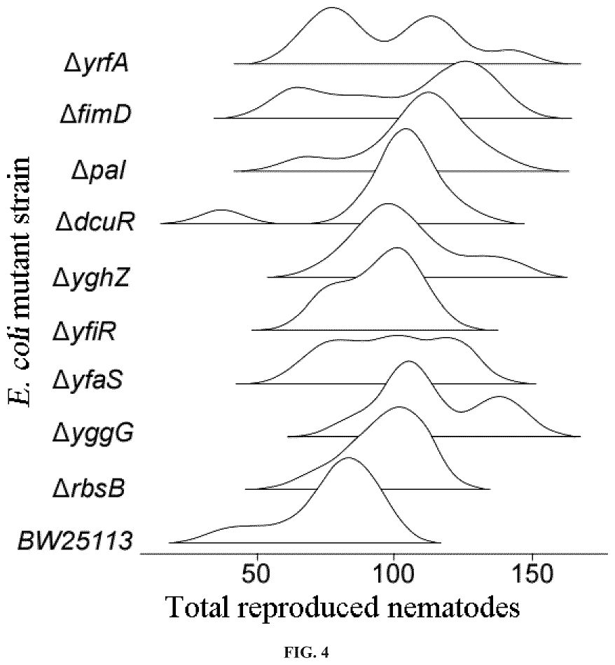

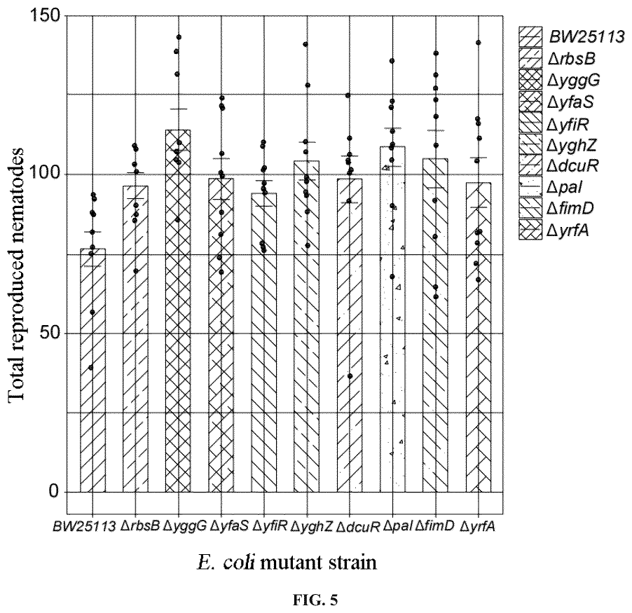

At 20° C. 1 mg/mL CTX was used to successfully construct the reproductive disorder model of C. elegans according to the method of Example 1, and then the reproductive disorder model was fed the strains of the Keio gene-knockout library of E. coli that were capable of improving the reproductive capacity of C. elegans. A control group and a treatment group were set up. The control group was fed a wild-type strain Bw25113 on NGM, and the treatment group was fed mutant strains of E. coli capable of improving reproductive capacity on NGM. One nematode was placed on each medium. The one nematode was picked to a new medium every day with a worm picker. After nematodes produced by this one nematode on the old medium grew to adults, the number of nematodes on NGM plates of the control group and the experimental group was counted until this one nematode no longer lay eggs. Referring to FIG. 4 and FIG. 5, it was seen that compared with the control group, the treatment group significantly improved the reproductive capacity of nematodes.

### Example 6

The wild-type N2 C. elegans was used to verify the strain with improved reproductive capacity selected in Example 4, and this wild-type N2 nematode was fed the strain in Example 4 for verification. The selected strain with improved reproductive capacity was cultured in a shaker at 37° C. for 12 h, centrifuged at 6,000 rpm and inoculated onto an NGM plate. Individual wild-type N2 nematode was picked into an NGM with E. coli mutants. Adults of N2 nematode was individually transferred to an NGM with a new E. coli mutant that could improve reproductive capacity until the C. elegans ceased to lay eggs. A total number of hatched eggs of each N2 nematode was calculated. Finally, a strain that was capable of significantly improving the reproductive capacity of nematodes was selected.

### Example 7

### A Method for Screening a Probiotic Capable of Promoting Reproductive Health

For the E. coli capable of improving the reproductive capacity of C. elegans selected in Example 5, a homologous strain of the probiotic Nissle 1917 was found, and a corresponding homologous gene was confirmed. The probiotic Nissle 1917 was modified with CRISPR/Cas9 gene editing technology, the corresponding homologous gene was knocked out, and then a modified probiotic was fed to C. elegans.

Treatment group: N2 larvae were placed in S broth containing CTX with a final concentration of 1 mg/mL for 72 h at the L1 stage to construct a reproductive disorder model of C. elegans, and then the reproductive disorder model of a single C. elegans was picked into an NGM with a new CRISPR/Cas9 gene-edited probiotic. N2 adults were individually transferred into a NGM with a new CRISPR/Cas9 gene-edited probiotic every day until the adults stopped laying eggs, and the number of hatched eggs of each N2 nematode was counted. Control group: N2 larvae were placed in S broth containing CTX with a final concentration of 1 mg/mL for 72 h at the L1 stage to construct a reproductive disorder model of C. elegans. The reproductive disorder model of single C. elegans was picked into a new NGM with the probiotic Nissle 1917. The N2 adults were individually transferred to a new NGM containing probiotic Nissle 1917 every day until the adults no longer lay eggs, and the number of egg hatching for each N2 nematode was counted. Compared with the control group, a probiotic in which the reproductive capacity of nematodes in the treatment group was significantly improved was the probiotic capable of promoting reproductive health.

In the present disclosure, a method for quickly constructing a reproductive disorder model of C. elegans is established, in favor of screening probiotics that are capable of promoting reproductive health. The method may screen probiotics that are capable of promoting reproductive health at a low cost, and has desirable market application values.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

1. A method for constructing a reproductive disorder model, comprising the following steps:
obtaining synchronized L1-stage Caenorhabditis elegans; and
putting the L1-stage C. elegans in an S broth containing 0.5 mg/mL to 2.0 mg/mL of cyclophosphamide (CTX) to allow culturing for 48 h to 96 h to complete model construction.

2. The method of claim 1, wherein the synchronized C. elegans is obtained by a sodium hypochlorite process.

3. The method of claim 1, wherein the synchronized C. elegans is cultured to hatch eggs to obtain the synchronized L1-stage C. elegans.

4. The method of claim 1, wherein the C. elegans is wild-type N2 C. elegans.

5. A method for screening a strain that promotes reproductive health, comprising feeding the reproductive disorder model constructed by the method of claim 1 with Escherichia coli strains and selecting E. coli strains capable of improving a reproductive capacity of the C. elegans.

6. (canceled)

7. The method of claim 5, wherein the E. coli strains comprise 3,985 kinds of E. coli strains in a Keio gene-knockout library of E. coli.

8. The method of claim 5, further comprising feeding the wild-type N2 C. elegans with the E. coli strains capable of improving the reproductive capacity of the C. elegans, and selecting an E. coli strain capable of significantly improving the reproductive capacity of the wild-type N2 C. elegans.

9. The method of claim 5, further comprising knocking out a homologous gene in a probiotic strain Nissle 1917 to obtain a probiotic strain capable of promoting the reproductive health, wherein the homologous gene corresponds to a knockout gene of the E. coli strain capable of improving the reproductive capacity of the C. elegans.

10. A method for verifying single gene deletion of E. coli in a reproductive system, comprising using the reproductive disorder model constructed by the method of claim 1.

11. The method of claim 5, wherein the synchronized C. elegans is obtained by a sodium hypochlorite process.

12. The method of claim 5, wherein the synchronized C. elegans is cultured to hatch eggs to obtain the synchronized L1-stage C. elegans.

13. The method of claim 5, wherein the C. elegans is wild-type N2 C. elegans.

14. The method of claim 5, wherein the synchronized C. elegans is obtained by a sodium hypochlorite process.

15. The method of claim 5, wherein the synchronized C. elegans is cultured to hatch eggs to obtain the synchronized L1-stage C. elegans.

16. The method of claim 5, wherein the C. elegans is wild-type N2 C. elegans.
